(19) United States
(12) Patent Application Publication
(10) Pub. No.: US 2025/0182876 A1
(43) Pub. Date: Jun. 5, 2025

(54) SYSTEMS AND METHODS FOR DISPLAYING, MONITORING AND SIMULATING HEMODYNAMIC SIGNALS, INDICATORS AND/OR OUTCOMES

(71) Applicant: Abiomed, Inc., Danvers, MA (US)

(72) Inventors: Christian Moyer, Danvers, MA (US); Ahmad El Katerji, Danvers, MA (US); Erik Ian Kroeker, Danvers, MA (US); Samir Jalloul, Danvers, MA (US); Steven Balk, Danvers, MA (US); Glen Fantuzzi, Danvers, MA (US); Tyler Valentine, Danvers, MA (US)

(21) Appl. No.: 18/969,891

(22) Filed: Dec. 5, 2024

Related U.S. Application Data
(60) Provisional application No. 63/706,156, filed on Oct. 11, 2024.
(60) Provisional application No. 63/606,555, filed on Dec. 5, 2023.

(51) Int. Cl. G16H 20/40; A61B 34/10; A61M 60/13; A61M 60/216; A61M 60/515; G16H 10/60; G16H 40/67

(52) U.S. Cl. CPC G16H 20/40; A61B 34/10; A61M 60/13; A61M 60/216; A61M 60/515; G16H 10/60; G16H 40/67; A61B 2034/105

(57) ABSTRACT

Methods and apparatus for simulating a state of a patient are provided. The method includes receiving a set of parameters associated with one or more of a medical device, a medical procedure, a medical treatment, or a medical condition, simulating, by at least one computer processor and using the set of parameters and a model representing characteristics of a patient, a state of the patient, and displaying, on a user interface associated with a computing device, a result of the simulating.

### CROSS-REFERENCE TO RELATED APPLICATIONS

This application claims the benefit under 35 U.S.C. § 119(e) to U.S. Provisional Patent Application No. 63/606,555, filed Dec. 5, 2023, and titled, “SYSTEMS AND METHODS FOR DISPLAYING, MONITORING AND SIMULATING HEMODYNAMIC AUXILIARY DISPLAY OF HEMODYNAMIC SIGNALS, INDICATORS AND/OR OUTCOMES,” and claims priority to U.S. Provisional Patent Application No. 63/706,156, filed Oct. 11, 2024, and titled “SYSTEMS AND METHODS FOR DISPLAYING, MONITORING AND SIMULATING HEMODYNAMIC AUXILIARY DISPLAY OF HEMODYNAMIC SIGNALS, INDICATORS AND/OR OUTCOMES,” the entire contents of each of which is incorporated by reference herein.

### FIELD OF THE INVENTION

This disclosure relates to techniques for simulating a patient state using medical information.

### BACKGROUND

Fluid pumps, such as blood pumps, are used in the medical field in a wide range of applications and purposes. An intravascular blood pump is a pump that can be advanced through a patient's vasculature, i.e., veins and/or arteries, to a position in the patient's heart or elsewhere within the patient's circulatory system. For example, an intravascular blood pump may be inserted via a catheter and positioned to span a heart valve. The intravascular blood pump is typically disposed at the end of the catheter. Once in position, the pump may be used to assist the heart and pump blood through the circulatory system and, therefore, temporarily reduce workload on the patient's heart, such as to enable the heart to recover after a heart attack. An exemplary intravascular blood pump is available from ABIOMED, Inc., Danvers, MA under the tradename Impella® heart pump.

An intravascular blood pump is typically connected to a respective external heart pump controller that controls the heart pump, such as motor speed, and collects and displays operational data about the blood pump, such as heart signal level, battery temperature, blood flow rate and plumbing integrity. An exemplary heart pump controller is available from ABIOMED, Inc. under the trade name Automated Impella Controller®. The controller may raise alarms when operational data values fall outside predetermined values or ranges, for example if a leak, suction, and/or pump malfunction is detected. The controller may include a video display screen upon which is displayed a graphical user interface configured to display the operational data and/or alarms.

### SUMMARY

Described herein are systems and methods for simulating a state of a patient using medical data associated with the patient. In some embodiments, a computer-implemented method of simulating a state of a patient is provided. The method includes receiving a set of parameters associated with one or more of a medical device, a medical procedure, a medical treatment, or a medical condition, simulating, by at least one computer processor and using the set of parameters and a model representing characteristics of a patient, a state of the patient, and displaying, on a user interface associated with a computing device, a result of the simulating.

In one aspect, simulating a state of the patient comprises providing the set of parameters as input to the model, and displaying a result of the simulating comprises displaying an indication of the state of the patient as values for the set of parameters are changed based on user input. In another aspect, the set of parameters includes one or more operating conditions of a medical device associated with the patient. In another aspect, the medical device comprises a cardiac support device, an extracorporeal membrane oxygenation device, a blood flow regulation device, a pacemaker, an infusion pump, and/or a dialysis machine. In another aspect, the set of parameters includes one or more outputs to optimize, simulating a state of the patient comprises determining one or more pathways or clinical adjustments to patient care that achieve the one or more outputs, and displaying a result of the simulating comprises displaying an indication of the one or more pathways or clinical adjustments.

In another aspect, the set of parameters comprises operation data associated with a medical device providing medical support to the patient, simulating a state of the patient comprises simulating changes to operation of the medical device, and the method further includes adjusting the operation of the medical device based, at least in part, on the result of the simulating. In another aspect, the set of parameters comprises dosage information for a pharmaceutical agent being provided to the patient, simulating a state of the patient comprises simulating changes to a dosage of the pharmaceutical agent to achieve a desired outcome included in the set of parameters, and displaying a result of the simulating comprises displaying an indication of a recommended dosage of the pharmaceutical agent determined based on the simulating. In another aspect, the patient is associated with multiple health issues, simulating a state of the patient comprises simulating a benefit of treating one or more of the multiple health issues to achieve a desired outcome included in the set of parameters, and displaying a result of the simulating comprises displaying a treatment recommendation based, at least in part, on the simulating. In another aspect, simulating a state of the patient further comprises determining a most cost effective and/or lowest risk combination of treatments to treat the patient.

In another aspect, receiving the set of parameters comprises receiving at least some parameters in the set of parameters from an electronic medical record associated with the patient. In another aspect, receiving the set of parameters comprises receiving at least some parameters in the set of parameters from a medical device associated with the patient. In another aspect, the medical device comprises a heart pump implanted in the patient. In another aspect, receiving the set of parameters comprises receiving at least some parameters in the set of parameters from an aggregator configured to aggregate data from a plurality of medical data sources. In another aspect, the plurality of medical data sources comprises a plurality of medical devices configured to provide medical support to the patient. In another aspect, the set of parameters includes information associated with a medical procedure, simulating a state of the patient comprises simulating an impact of performing the medical procedure on the patient on a cardiac health of the patient, and displaying a result of the simulating comprises displaying a recommendation of whether to perform the medical procedure. In another aspect, the medical procedure includes one or more of a staged percutaneous coronary intervention, a coronary artery bypass graft surgery, a degenerative mitral valve surgery, or a transcatheter aortic valve replacement. In another aspect, displaying, on a user interface associated with a computing device, a result of the simulating comprises transmitting the result of the simulating to a remote device management system configured to provide the user interface.

In some embodiments, a computing system is provided. The computing system includes a communication interface configured to receive a set of parameters associated with one or more of a medical device, a medical procedure, a medical treatment, or a medical condition, and at least one computer processor. The at least one computer processor is configured to simulate using the set of parameters and a model representing characteristics of a patient, a state of the patient; and display, on a user interface associated with a computing device, a result of the simulating.

In one aspect, the at least one computer processor is configured to provide the set of parameters as input to the model, and display a result of the simulating by displaying an indication of the state of the patient as values for the set of parameters are changed based on user input. In another aspect, the set of parameters include one or more operating conditions of a medical device associated with the patient. In another aspect, the medical device comprises a cardiac support device, an extracorporeal membrane oxygenation device, a blood flow regulation device, a pacemaker, an infusion pump, and/or a dialysis machine.

In another aspect, the set of parameters includes one or more outputs to optimize, and the at least one computer processor is configured to simulate a state of the patient by determining one or more pathways or clinical adjustments to patient care that achieve the one or more outputs, and display a result of the simulating by displaying an indication of the one or more pathways or clinical adjustments. In another aspect, the set of parameters comprises operation data associated with a medical device providing medical support to the patient, and the at least one computer processor is configured to simulate a state of the patient by simulating changes to operation of the medical device, and adjust the operation of the medical device based, at least in part, on the result of the simulating. In another aspect, the set of parameters comprises dosage information for a pharmaceutical agent being provided to the patient, and the at least one computer processor is configured to simulate a state of the patient comprises by simulating changes to a dosage of the pharmaceutical agent to achieve a desired outcome included in the set of parameters, and display a result of the simulating by displaying an indication of a recommended dosage of the pharmaceutical agent determined based on the simulating.

In another aspect, the patient is associated with multiple health issues, and the at least one computer processor is configured to simulate a state of the patient by simulating a benefit of treating one or more of the multiple health issues to achieve a desired outcome included in the set of parameters, and display a result of the simulating by displaying a treatment recommendation based, at least in part, on the simulating. In another aspect, the at least one computer processor is configured to simulate a state of the patient further by determining a most cost effective and/or lowest risk combination of treatments to treat the patient.

In another aspect, the communication interface is configured to receive at least some parameters in the set of parameters from an electronic medical record associated with the patient. In another aspect, the communication interface is configured to receive at least some parameters in the set of parameters from a medical device associated with the patient. In another aspect, the medical device comprises a heart pump implanted in the patient. In another aspect, the communication interface is configured to receive at least some parameters in the set of parameters from an aggregator configured to aggregate data from a plurality of medical data sources. In another aspect, the plurality of medical data sources comprises a plurality of medical devices configured to provide medical support to the patient.

In another aspect, the set of parameters includes information associated with a medical procedure, and the at least one computer processor is configured to simulate a state of the patient by simulating an impact of performing the medical procedure on the patient on a cardiac health of the patient, and display a result of the simulating by displaying a recommendation of whether to perform the medical procedure. In another aspect, the medical procedure includes one or more of a staged percutaneous coronary intervention, a coronary artery bypass graft surgery, a degenerative mitral valve surgery, or a transcatheter aortic valve replacement. In another aspect, the at least one computer processor is configured to display, on a user interface associated with a computing device, a result of the simulating by transmitting the result of the simulating to a remote device management system configured to provide the user interface.

### BRIEF DESCRIPTION OF DRAWINGS

FIG. 1 shows an illustrative cardiac support device that may be used to generate monitored data for use with some embodiments.

FIG. 2 shows a flowchart of a process according to some embodiments.

FIG. 3A shows an architecture of a system for performing a simulation according to some embodiments.

FIG. 3B shows another architecture of a system for performing a simulation according to some embodiments.

FIG. 3C shows another architecture of a system for performing a simulation according to some embodiments.

FIG. 3D shows another architecture of a system for performing a simulation according to some embodiments.

### DETAILED DESCRIPTION

As is known, blood pumps may be implanted into a patient's heart to assist the heart and pump blood through the circulatory system and temporarily reduce workload on the patient's heart, such as to enable the heart to recover after an acute injury (e.g., after a heart attack). Such blood pumps may include one or more sensors for measuring one or more parameters relating to the blood pump and/or to the patient. In some embodiments, the one or more parameters relating to the blood pump and/or the patient may be used by a clinician to determine the appropriate course of treatment for the patient (e.g., whether to increase or decrease support level and/or the length of support).

The inventors have appreciated the benefits of having a system that would allow a clinician to simulate the effects of potential interventions available to a patient and/or the effects of patient conditions with an existing intervention. For example, what would happen if a new device was inserted in addition to an implanted pump? Or, as another example, what would happen if the implanted device was removed? Would the patient's condition improve or worsen in such situations. Embodiments disclosed herein relate to a system that enable a clinician to create desired simulations to evaluate patient outcomes and determine which, if any, intervention would be beneficial to the patient.

As is known, cardiovascular systems can be modeled as an electrical circuit, referred to herein as a “lumped parameter model.” Like any electrical circuit with time-varying components (e.g., capacitors, inductors, diodes, etc.), the model may include a set of differential equations and constitutive relationships, where each equation and relationship can be further defined through coefficients and parameters. Such coefficients, parameters, relationships, etc., may be optimized to represent a certain cardiovascular state, and can be modified to simulate a change in cardiovascular state, caused by a physiologic event (e.g., acute myocardial infarction (AMI)) or an intervention (e.g., a device implant). The model may be deterministic and fixed, such that one set of parameters may represent one cardiovascular state.

In some embodiments disclosed herein, a system may include a model configured to represent some or all of the above, in greater detail, with multiple organ systems, devices, and relationships captured in the single model. In some embodiments, the system, may be optimized for a canonical state (e.g., “normal”, “heart failure”, “AMI” etc.) and may not necessarily represent an individual patient. Such systems may be further refined by optimizing model parameters to represent an individual patient and/or an individual cardiovascular state. In some instances, to execute such an optimization, sufficient input data (e.g., physiologic signals) must be available. As described herein, the input data may include data relating to a specific condition (or conditions) being evaluated by a clinician and/or relating to a specific device (e.g., a heart pump) being implanted.

In some embodiments, once the system is optimized for an individual patient, it can then be used to simulate an effect of a potential intervention and/or patient condition. In some embodiments, a simulation of a “what if” scenario that may be deterministic and may use an identical digital copy, also referred to herein as a “digital twin” may be used as a starting point to evaluate such scenarios.

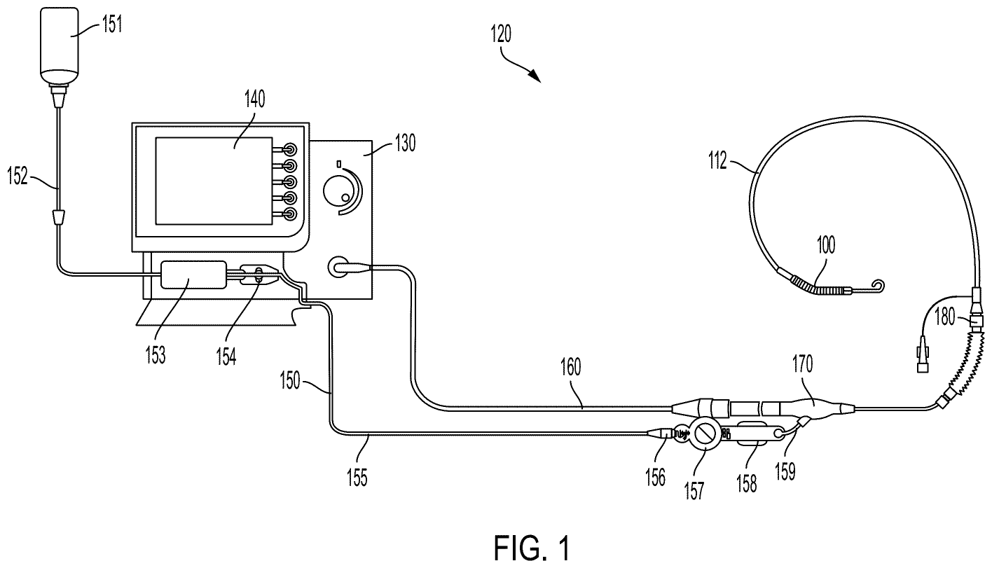

Turning now to the figures, FIG. 1 shows a heart pump 110 according to one embodiment of the present disclosure. As will be appreciated, a circulatory support device (also referred to herein as a “heart pump” or simply a “pump”) may include a percutaneous, catheter-based device that provides hemodynamic support to the heart of a patient. As shown in FIG. 1, heart pump 110 may form part of a cardiac support system 100. Cardiac support system 100 also may include a controller 130 (e.g., an Automated Impella Controller®, referred to herein as an “AIC,” from ABIOMED, Inc., Danvers, Mass.), a display 140, a purge subsystem 150, a connector cable 160, a plug 170, and a repositioning unit 180. As shown, controller 130 may include display 140. Controller 130 may be configured to monitor and control operation of heart pump 110. During operation, purge subsystem 150 may be configured to deliver a purge fluid to heart pump 110 through catheter tube 117 to prevent blood from entering the motor (not shown) of the heart pump. In some implementations, the purge fluid is a dextrose solution (e.g., 5% dextrose in water with 25 or 50 IU/mL of heparin, although the solution need not include heparin in all embodiments). Connector cable 160 may provide an electrical connection between heart pump 110 and controller 130. Plug 170 may connect catheter tube 117, purge subsystem 150, and connector cable 160. In some implementations, plug 170 includes a storage device (e.g., a memory) configured to store, for example, operating parameters to facilitate transfer of the patient to another controller if needed. Repositioning unit 180 may be used to reposition heart pump 110 in the patient's heart.

As shown in FIG. 1, in some embodiments, the cardiac support system 100 may include a purge subsystem 150 having a container 151, a supply line 152, a purge cassette 153, a purge disc 154, purge tubing 155, a check valve 156, a pressure reservoir 157, an infusion filter 158, and a sidearm 159. Container 151 may, for example, be a bag or a bottle. As will be appreciated, in other embodiments the cardiac support system 100 may not include a purge subsystem. In some embodiments, a purge fluid may be stored in container 151. Supply line 152 may provide a fluidic connection between container 151 and purge cassette 153. Purge cassette 153 may control how the purge fluid in container 151 is delivered to heart pump 110. For example, purge cassette 153 may include one or more valves for controlling a pressure and/or flow rate of the purge fluid. Purge disc 154 may include one or more pressure and/or flow sensors for measuring a pressure and/or flow rate of the purge fluid. As shown, controller 130 may include purge cassette 153 and purge disc 154. Purge tubing 155 may provide a fluidic connection between purge disc 154 and check valve 156. Pressure reservoir 157 may provide additional filling volume during a purge fluid change. In some implementations, pressure reservoir 157 includes a flexible rubber diaphragm that provides the additional filling volume by means of an expansion chamber. Infusion filter 158 may help prevent bacterial contamination and air from entering catheter tube 117. Sidearm 159 may provide a fluidic connection between infusion filter 158 and plug 170. Although shown as having separate purge tubing and connector cable, it will be appreciated that in some embodiments, the cardiac support system 100 may include a single connector with both fluidic and electric lines connectable to the controller 130.

During operation, controller 130 may be configured to receive measurements from one or more pressure sensors (not shown) included as a portion of heart pump 110 and purge disc 154. Controller 130 may also be configured to control operation of the motor (not shown) of the heart pump 110 and purge cassette 153. As noted herein, controller 130 may be configured to control and measure a pressure and/or flow rate of a purge fluid via purge cassette 153 and purge disc 154. During operation, after exiting purge subsystem 150 through sidearm 159, the purge fluid may be channeled through purge lumens (not shown) within catheter tube 117 and plug 170. Sensor cables (not shown) within catheter tube 117, connector cable 160, and plug 170 may provide an electrical connection between components of the heart pump 110 (e.g., one or more pressure sensors) and controller 130. Motor cables (not shown) within catheter tube 117, connector cable 160, and plug 170 may provide an electrical connection between the motor of the heart pump 110 and controller 130. During operation, controller 130 may be configured to receive measurements from one or more pressure sensors of the heart pump 110 through the sensor cables (e.g., optical fibers) and to control the electrical power delivered to the motor of the heart pump 110 through the motor cables. By controlling the power delivered to the motor of the heart pump 110, controller 130 may be operable to control the speed of the motor.

Various modifications can be made to cardiac support system 100 and one or more of its components. For instance, one or more additional sensors may be added to heart pump 100. In another example, a signal generator may be added to heart pump 100 to generate a signal indicative of the rotational speed of the motor of the heart pump 110. As another example, one or more components of cardiac support system 100 may be separated. For instance, display 140 may be incorporated into another device in communication with controller 130 (e.g., wirelessly or through one or more electrical cables).

A heart pump (e.g., heart pump 110) may include a pressure sensor (e.g., an optical pressure sensor) configured to detect a pressure within the aorta of a patient's heart when the heart pump is properly positioned. The pressure signal sensed by the pressure sensor may be used, at least in part, to determine correct positioning of the heart pump within the patient's heart and/or to determine a blood flow rate through the heart pump when in operation. For instance, the pressure signal may be used in combination with a motor current signal received from a motor current sensor (not shown) and a set of stored values to determine a flow rate of blood through the heart pump. The differential pressure across the aortic valve may also indirectly be determined based on the pressure signal measuring the pressure in the aorta and the set of stored values.

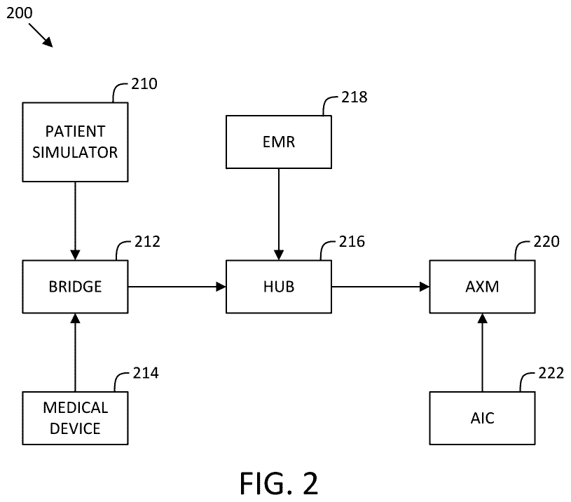

FIG. 2 illustrates a flowchart 200 according to some embodiments of the present disclosure. As shown in this view, the patient simulator 210 (which may include the “digital twin” model) may send information to a bridge 212, which may aggregate data from the patient simulator 210 and one or more medical devices 214 (e.g., an ECMO machine) also connected to the bridge 212. The bridge 212 may then send the received data to a hub 216. As shown in this view, the hub 216 may be configured to also receive electronic medical records (EMR) 218. The hub 216 may then send data to an auxiliary monitor (AXM) 220, which can display the data to a clinician (e.g., data from the patient simulator 210 and/or health information). As will be appreciated, such health information may be deidentified before being sent to the AXM 220. In some embodiments, the AXM 220 may be connected to a controller 222 of a medical device (e.g., a controller (AIC) of a blood pump). As will be appreciated, there may be other suitable connections (e.g., other medical devices connected to the AXM instead of to the hub) in some embodiments.

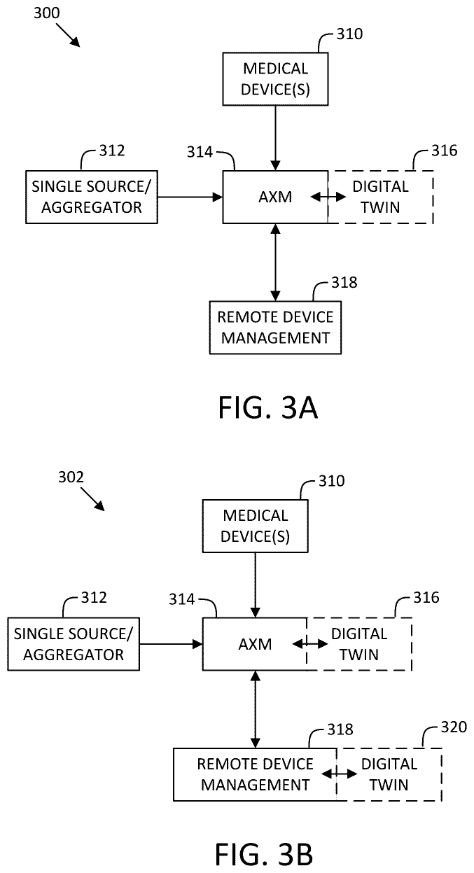

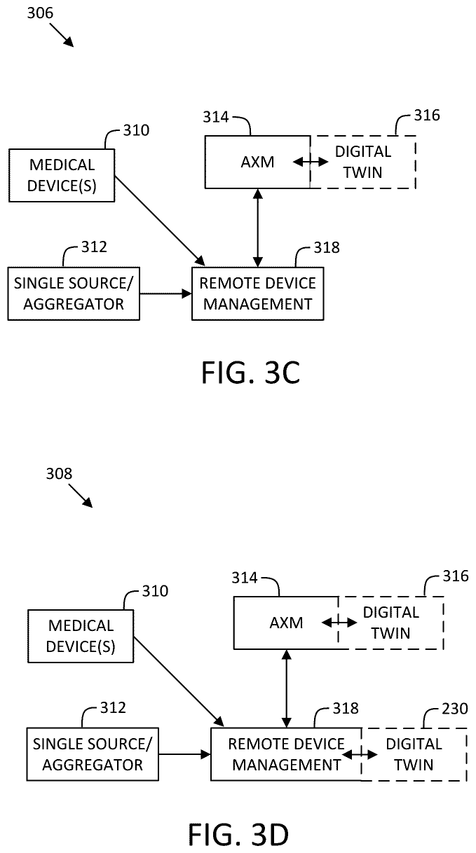

As described herein, in some embodiments, a digital twin, which may be a digital model representing characteristics of an individual patient, may be used to simulate one or more “what-if” scenarios regarding the patient. FIG. 3A illustrates an architecture 300, in accordance with some embodiments. In architecture 300, digital twin 316 is associated with (e.g., included as a portion of or in communication with) auxiliary monitor 314. As shown in architecture 300, auxiliary monitor 314 may receive data from one or more medical devices 310. Examples of medical devices 310 include, but are not limited to, mechanical circulatory support (MCS) devices (e.g., an Impella® heart pump available from ABIOMED, Inc.), ventilators, ECMO devices, blood flow regulation devices, pacemakers, infusion pumps, and dialysis machines. Additionally or alternatively, auxiliary monitor 314 may receive data from a single source 312 that aggregates data from multiple medical devices (e.g., multiple heart pumps, one or more heart pumps and one or more blood flow regulation devices, etc.). Auxiliary monitor 314 may also be communicatively coupled to remote device management system 318. An example of remote device management system 318 is the Impella Connect® system available from ABIOMED, Inc., which is configured to enable clinicians and other users to remotely view data (e.g., summary data, real-time data, video stream data, etc.) associated with medical devices (e.g., medical devices 310) such as an Impella® heart pump. As shown in FIG. 3A, in some embodiments the communication between auxiliary monitor 314 and remote device management system 318 may be bidirectional such that a user may interact with a device (e.g., a laptop computer, a smartphone, etc.) coupled to remote device management system 318 to request performance of a simulation using digital twin 316 associated with auxiliary monitor 314, and the results of the simulation may be sent from auxiliary monitor 314 to the remote device management system 318, which may be configured to display the results on the user's device.

FIG. 3B illustrates an architecture 302, in accordance with some embodiments. Architecture 302 includes some components similar to architecture 300, and additionally includes a second digital twin 320 associated with (e.g., included as a portion of or in communication with) remote device management system 318. Inclusion of second digital twin 320 may enable the devices connected to the remote device management system 318 to request and perform simulations using the second digital twin 320 even when the remote device management system 318 is not communicatively coupled to (e.g., not in network communication with) auxiliary monitor 314. In some embodiments, a first digital twin 316 associated with auxiliary monitor 314 and the second digital twin 320 associated with remote device management system 318 may be the same (e.g., the second digital twin 320 may be a copy of the first digital twin 316).

FIG. 3C illustrates an architecture 306, in accordance with some embodiments. In architecture 306, data from medical device(s) 310 and/or single source 312 are provided to remote device management system 318 rather than being provided to auxiliary monitor 314, as described in connection with architectures 300 and 302 shown in FIGS. 3A and 3B, respectively. FIG. 3D illustrates an architecture 308, in accordance with some embodiments. Architecture 308 includes some components similar to architecture 306, and additionally includes a second digital twin 320 associated with (e.g., included as a portion of or in communication with) remote device management system 318. Inclusion of second digital twin 320 may enable auxiliary monitor 314 to perform simulations using second digital twin 320 even when the remote device management system 318 is not communicatively coupled to (e.g., not in network communication with) auxiliary monitor 314. In some embodiments, a first digital twin 316 associated with auxiliary monitor 314 and the second digital twin 320 associated with remote device management system 318 may be the same (e.g., the second digital twin 320 may be a copy of the first digital twin 316).

Although a digital twin is shown in architectures 300, 302, 306 and 308 as being associated with auxiliary monitor 314 and/or remote device management system 318, it should be appreciated that a digital twin generated in accordance with the techniques described herein may be associated with any suitable component of a computing architecture. As an example, in some embodiments, a simulation using a digital twin may be performed using a web interface (e.g., via a web browser) when the digital twin in located on a cloud-computing/network device. In some embodiments, the digital twin assessments may be implemented on an app specific to performing a particular diagnosis. In a healthcare setting, the digital twin assessments may be implemented on a rounding computer (e.g., a tablet computer taken on rounds), within one or more healthcare applications, such as an electronic medical record, or on any other computer within the healthcare setting, an example of which includes being implemented on a nursing station computer.

According to some embodiments, a method includes creating a digital twin/“patient specific” cardiovascular simulation. In some embodiments, the method includes running a stepwise optimization, where coefficients for a first subset of differential equations included in the simulation are allowed to vary while coefficients for a second subset of differential equations included in the simulation are held constant (e.g., the coefficients are fixed). The coefficients that are allowed to vary may be optimized to minimize the error between the clinical inputs and the simulated variables.

The method may further include simulating the possible clinical outputs given the distribution of each known parameter in the system of differential equations and producing a distribution of clinical outputs. Then, given a set of clinical outputs representing a specific patient, the method may include computing all possible input parameter values that produce the set of clinical outputs, averaging those possible input parameters, and iterating the input-output simulation to further refine input parameters until an optimum is achieved representing the individual patient state.

The method may further include leveraging the presence of a mechanical support device or pharmacologic support medication to create multiple clinical states, for instance by varying the dose or support level of the mechanical support device. The method may then include using these multiple observable clinical states with known changes to the support device or medication and assume that a subset of input parameters remained fixed during this intervention to improve the optimization of the parameters.

The method may further include running a sequential optimization where all model parameters remained fixed, and a first parameter is allowed to vary to achieve as close as possible, the clinical inputs of a given patient. In some embodiments, the first parameter is then fixed at that new state, and a second parameter is allowed to vary in a similar fashion to achieve a closer optimized state. The second parameter is then fixed and sequentially the system moves through each parameter, either once or using multiple cycles until a global optimum is achieved.

In some embodiments, a machine learning encoder-decoder relationship may be built, where many simulations are created and stored (e.g., simulated parameter sets that produce simulated clinical outputs), the clinical outputs may then be used to train an encoder-decoder model, where the clinical inputs are encoded into a latent space, which is then decoded to best match the model parameter set. Once this is created, the encoder may then be extracted and stored as a best presentation of the model relationships. When presented with a new, patient specific set of clinical inputs, the inputs may then be pushed through the same encoder-decoder relationship to estimate the patient model parameters. In some embodiments, this may be considered a “many to many” encoder-decoder design.

A Bayesian optimization approach that allows for multiple model parameter sets that can represent an individual patient, with assigned probabilities to each of those parameter sets (e.g., probability that the individual patient has those underlying model parameters) also may be used. In some embodiments, this may then be translated into what if scenarios, where multiple “patients” (in a probabilities framework) are assessed through the same “what if” scenario, where potential outputs are weighted by those same probabilities.

In some embodiments, a digital twin may be used as a screener tool to determine whether a patient would benefit from use of a mechanical circulatory support (MCS) device. For instance, the digital twin may have access to information from patients with and without MCS devices to evaluate whether an optimization output from a simulation indicates that a patient would benefit from an MCS device. In some embodiments, a digital twin may be used to simulate general patient management (e.g., adding volume to the patient's cardiac system).

In some embodiments, a digital twin may be used to simulate how one or more surgical and/or cardiac catheterization lab (CCL) procedures may impact a patient's cardiac health. Examples of surgical and/or CCL procedures that may be included in a simulation include, but are not limited to, staged percutaneous coronary intervention (PCI), coronary artery bypass graft (CABG) surgery with or without degenerative mitral valve (DMV) surgery (e.g., combined DMV+CABG surgery), and transcatheter aortic valve replacement (TAVR) for patients with or without bicuspid aortic valve (BAV) disease when combined or separate from a PCI procedure.

In some embodiments, a user may be provided (e.g., via a user interface) with an option to perform different types of simulations using a digital twin. In a first (e.g., basic) simulation, a user may be able to change inputs to a digital twin and observe how changing the different inputs is reflected in the output of the digital twin model after simulation using the inputs. In a second (e.g., output-driven) simulation, a user may provide a set of inputs with specific outputs to optimize in the simulation, and a simulation may be performed to provide one or more “pathways” or clinical adjustments to obtain the specific outputs. In a third (e.g., full) type of simulation, the user may request a full optimization and the digital twin may optimize all variables based on a set of inputs to achieve desired output.

In some embodiments, a digital twin may be used to perform closed loop patient management. For example, a user may request a simulation with “full optimization”, and the system may adjust the operation of devices (e.g., heart pump(s)) and or pharmacological agents to achieve the desired results. Any suitable pharmacological agents may be considered when performing the simulation, examples of which include, but are not limited to, inotropes, vasodepressors, diuretics/volume management agents, blood thinning agents, and heart rhythm related medications.

In some embodiments, a digital twin may be used to evaluate whether a patient with multiple diseases and/or multiple health issues would benefit from different treatment regiments. For instance, a simulation using the digital twin may enable a determination of the most cost-effective and/or lowest risk combination of treatments to treat a patient. As an example, a patient may present with three primary health issues, and simulations may be performed using a digital twin to observe the benefit (e.g., based on the outputs of the simulation) of treating any one of the issues, any two of the issues, or all three of the issues. Based on the outputs of the simulations it may be determined that treating all three issues may provide the patient with only marginal (or no) benefit compared with treating a single or a combination of two of the issues, which may result is more cost-effective treatment of the patient while still achieving a goal health state.

In some embodiments, a digital twin may be used to modify an original treatment plan during the course of the treatment. For example, a physician may use the digital twin to perform simulations during a treatment plan and based on the output of the simulation may adjust, alter, or optimize the treatment plan to improve patient outcomes.

In some embodiments, a digital twin may require a set of values for all input variables to perform an optimization/simulation. In configurations where all of the variables/inputs are continuously available, there may be no issue in performing the simulations. In select instances where fewer than all of the variables/inputs are continuously available, the system may use the most recently available data for the missing variables/inputs or may query a user to manually input the missing values. In some embodiments where values for missing variables are provided (e.g., either based on previous values or via user input), the output(s) of the optimization/simulation may be provided with a notation/warning indicating that the output(s) were determined using incomplete information.

In some embodiments a method includes assessing the robustness of a digital twin/“patient specific” model. The method may include running a sensitivity analysis, where an optimized model allows a single parameter to vary to understand how much residual error can be introduced by that parameter. Parameter(s) determined to have the largest sensitivity for a given optimized patient state may then be reassessed following mechanical or pharmacological interventions that slightly alter the state without impacting that parameter, thus allowing a model optimization to build confidence where it is most necessary.

In some embodiments, a method of model optimization can be challenged by running “leave one out” scenarios, where an available clinical input is artificially withheld during a model optimization routine. In such embodiments, the model may be allowed to optimize, then the withheld clinical input may be assessed for residual error vs. the simulated output of that same clinical variable. Clinical variables deemed the most sensitive to this test may be reassessed or supported by additional clinical measurements to improve an optimization.

In some embodiments, a method includes leveraging a digital twin to simulate a “what if”' scenario (e.g., simulation of potential intervention). In some embodiments, the same encoder-decoder framework described above may be utilized but may now be created for variable states of mechanical support. In this framework, the encoder-decoder may be built on all the possible mechanical/pharmacological support scenarios, given a fixed model input or a subset of potential model inputs that represent the patient (e.g., the “digital twin”). The encoder may read in all the potential mechanical support scenarios and decode them against simulated output to best represent the what if scenario, given the starting patient. In some embodiments, the method may include applying the Bayesian probabilities described above, where they are carried forward to multiple what-if scenarios. In some embodiments, an update to the model design that accounts for a patient response to a model input (e.g., a previously optimized model parameter adapts to the presence of mechanical or pharmacological support), in this scenario a previously fixed parameter, may be allowed to float or vary, in order to simulate all the potential outputs from a “what if” scenario.

Having thus described several aspects and embodiments of the technology set forth in the disclosure, it is to be appreciated that various alterations, modifications, and improvements will readily occur to those skilled in the art. Such alterations, modifications, and improvements are intended to be within the spirit and scope of the technology described herein. For example, those of ordinary skill in the art will readily envision a variety of other means and/or structures for performing the function and/or obtaining the results and/or one or more of the advantages described herein, and each of such variations and/or modifications is deemed to be within the scope of the embodiments described herein. Those skilled in the art will recognize or be able to ascertain using no more than routine experimentation, many equivalents to the specific embodiments described herein. It is, therefore, to be understood that the foregoing embodiments are presented by way of example only and that, within the scope of the appended claims and equivalents thereto, inventive embodiments may be practiced otherwise than as specifically described. In addition, any combination of two or more features, systems, articles, materials, kits, and/or methods described herein, if such features, systems, articles, materials, kits, and/or methods are not mutually inconsistent, is included within the scope of the present disclosure.

The above-described embodiments can be implemented in any of numerous ways. One or more aspects and embodiments of the present disclosure involving the performance of processes or methods may utilize program instructions executable by a device (e.g., a computer, a processor, or other device) to perform, or control performance of, the processes or methods. In this respect, various inventive concepts may be embodied as a computer readable storage medium (or multiple computer readable storage media) (e.g., a computer memory, one or more floppy discs, compact discs, optical discs, magnetic tapes, flash memories, circuit configurations in Field Programmable Gate Arrays or other semiconductor devices, or other tangible computer storage medium) encoded with one or more programs that, when executed on one or more computers or other processors, perform methods that implement one or more of the various embodiments described above. The computer readable medium or media can be transportable, such that the program or programs stored thereon can be loaded onto one or more different computers or other processors to implement various ones of the aspects described above. In some embodiments, computer readable media may be non-transitory media.

The above-described embodiments of the present technology can be implemented in any of numerous ways. For example, the embodiments may be implemented using hardware, software or a combination thereof. When implemented in software, the software code can be executed on any suitable processor or collection of processors, whether provided in a single computer or distributed among multiple computers. It should be appreciated that any component or collection of components that perform the functions described above can be generically considered as a controller that controls the above-described function. A controller can be implemented in numerous ways, such as with dedicated hardware, or with general purpose hardware (e.g., one or more processor) that is programmed using microcode or software to perform the functions recited above and may be implemented in a combination of ways when the controller corresponds to multiple components of a system.

Further, it should be appreciated that a computer may be embodied in any of a number of forms, such as a rack-mounted computer, a desktop computer, a laptop computer, or a tablet computer, as non-limiting examples. Additionally, a computer may be embedded in a device not generally regarded as a computer but with suitable processing capabilities, including a Personal Digital Assistant (PDA), a smartphone or any other suitable portable or fixed electronic device.

Also, a computer may have one or more input and output devices. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards, and pointing devices, such as mice, touch pads, and digitizing tablets. As another example, a computer may receive input information through speech recognition or in other audible formats.

Such computers may be interconnected by one or more networks in any suitable form, including a local area network or a wide area network, such as an enterprise network, and intelligent network (IN) or the Internet. Such networks may be based on any suitable technology and may operate according to any suitable protocol and may include wireless networks, wired networks or fiber optic networks.

Also, as described, some aspects may be embodied as one or more methods. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

1. A computer-implemented method of simulating a state of a patient, the method comprising:
receiving a set of parameters associated with one or more of a medical device, a medical procedure, a medical treatment, or a medical condition;
simulating, by at least one computer processor and using the set of parameters and a model representing characteristics of a patient, a state of the patient; and
displaying, on a user interface associated with a computing device, a result of the simulating.

2. The method of claim 1, wherein
simulating a state of the patient comprises providing the set of parameters as input to the model, and
displaying a result of the simulating comprises displaying an indication of the state of the patient as values for the set of parameters are changed based on user input.

3. The method of claim 2, wherein the set of parameters includes one or more operating conditions of a medical device associated with the patient.

4. The method of claim 3, wherein the medical device comprises a cardiac support device, an extracorporeal membrane oxygenation device, a blood flow regulation device, a pacemaker, an infusion pump, and/or a dialysis machine.

5. The method of claim 1, wherein
the set of parameters includes one or more outputs to optimize,
simulating a state of the patient comprises determining one or more pathways or clinical adjustments to patient care that achieve the one or more outputs, and
displaying a result of the simulating comprises displaying an indication of the one or more pathways or clinical adjustments.

6. The method of claim 1, wherein
the set of parameters comprises operation data associated with a medical device providing medical support to the patient,
simulating a state of the patient comprises simulating changes to operation of the medical device, and
the method further comprises adjusting the operation of the medical device based, at least in part, on the result of the simulating.

7. The method of claim 1, wherein
the set of parameters comprises dosage information for a pharmaceutical agent being provided to the patient,
simulating a state of the patient comprises simulating changes to a dosage of the pharmaceutical agent to achieve a desired outcome included in the set of parameters, and
displaying a result of the simulating comprises displaying an indication of a recommended dosage of the pharmaceutical agent determined based on the simulating.

8. The method of claim 1, wherein
the patient is associated with multiple health issues,
simulating a state of the patient comprises simulating a benefit of treating one or more of the multiple health issues to achieve a desired outcome included in the set of parameters, and
displaying a result of the simulating comprises displaying a treatment recommendation based, at least in part, on the simulating.

9. The method of claim 8, wherein simulating a state of the patient further comprises determining a most cost effective and/or lowest risk combination of treatments to treat the patient.

10. The method of claim 1, wherein receiving the set of parameters comprises receiving at least some parameters in the set of parameters from an electronic medical record associated with the patient.

11. The method of claim 1, wherein receiving the set of parameters comprises receiving at least some parameters in the set of parameters from a medical device associated with the patient.

12. The method of claim 11, wherein the medical device comprises a heart pump implanted in the patient.

13. The method of claim 1, wherein receiving the set of parameters comprises receiving at least some parameters in the set of parameters from an aggregator configured to aggregate data from a plurality of medical data sources.

14. The method of claim 13, wherein the plurality of medical data sources comprises a plurality of medical devices configured to provide medical support to the patient.

15. The method of claim 1, wherein
the set of parameters includes information associated with a medical procedure,
simulating a state of the patient comprises simulating an impact of performing the medical procedure on the patient on a cardiac health of the patient, and
displaying a result of the simulating comprises displaying a recommendation of whether to perform the medical procedure.

16. The method of claim 15, wherein the medical procedure includes one or more of a staged percutaneous coronary intervention, a coronary artery bypass graft surgery, a degenerative mitral valve surgery, or a transcatheter aortic valve replacement.

17. The method of claim 1, wherein displaying, on a user interface associated with a computing device, a result of the simulating comprises transmitting the result of the simulating to a remote device management system configured to provide the user interface.

18. A computing system, comprising:
a communication interface configured to receive a set of parameters associated with one or more of a medical device, a medical procedure, a medical treatment, or a medical condition; and
at least one computer processor configured to:
simulate using the set of parameters and a model representing characteristics of a patient, a state of the patient; and
display, on a user interface associated with a computing device, a result of the simulating.

19. The computing system of claim 18, wherein the at least one computer processor is configured to:
provide the set of parameters as input to the model; and
display a result of the simulating by displaying an indication of the state of the patient as values for the set of parameters are changed based on user input.

20-21. (canceled).

22. The computing system of claim 18, wherein
the set of parameters includes one or more outputs to optimize, and
the at least one computer processor is configured to:
simulate a state of the patient by determining one or more pathways or clinical adjustments to patient care that achieve the one or more outputs; and
display a result of the simulating by displaying an indication of the one or more pathways or clinical adjustments.

23-34. (canceled).
